(19) United States
(12) Patent Application Publication
(10) Pub. No.: US 2026/0033749 A1
(43) Pub. Date: Feb. 5, 2026

(54) HEARING TESTING SYSTEM AND METHOD

(71) Applicant: Delta Electronics, Inc., Taoyuan City (TW)

(72) Inventors: Yu-Hsuan Hu, Taoyuan City (TW); Shih-Chieh Chien, Taoyuan City (TW); Jiang-Chun Chen, Taoyuan City (TW); Chi-Yueh Lin, Taoyuan City (TW); Fu-Kai Chuang, Taoyuan City (TW)

(21) Appl. No.: 19/221,993

(22) Filed: May 29, 2025

(30) Foreign Application Priority Data
Jul. 30, 2024 (CN) 202411031022.4

(51) Int. Cl. A61B 5/12

(52) U.S. Cl. CPC A61B 5/123

(57) ABSTRACT

The present disclosure provides a hearing testing system and method, the hearing testing system includes a playback module and a detection module. The playback module includes an intensity-level adjustment module connecting to a player for setting the intensity level of a test sound, and the player plays the test sound to a test ear of a user. The test sound includes a plurality of pure tones having different decibels or frequencies. The detection module is in communication with the playback module, and the user generates a single test response corresponding to the plurality of pure tones having different decibels or frequencies. The test response is that the user inputs the number of pure tones heard into the detection module.

### CROSS-REFERENCE TO RELATED APPLICATION

This application claims priority to China Patent Application No. 202411031022.4, filed on Jul. 30, 2024, the entire contents of which are incorporated herein by reference for all purposes.

### FIELD OF THE INVENTION

The present disclosure relates to a hearing testing system and method, and more particularly to a hearing testing system and method capable of reducing testing time.

### BACKGROUND OF THE INVENTION

In the conventional hearing testing techniques, pure tone audiometry (PTA) is recognized as an accurate testing way. In the process of PTA, a single pure tone is played to a test subject through a player, and a professional records the response of that whether the test subject hears the single pure tone. The playing and response process is repeated several times with different frequencies and intensity levels of single pure tones for obtaining a complete hearing report of the test subject. However, the mentioned testing way requires a professional to be present for assisting in recording the test subject's responses and adjusting the frequencies and intensity levels of the single pure tones played by the player. Moreover, since the test subject needs to respond to each single pure tone every time, the testing time would be really long.

The conventional hearing testing techniques are applied in mobile apps, by comparing the test subject's testing data at two frequencies, the test subject's hearing status at other frequencies can be inferred. Therefore, the test subject may complete the hearing testing independently through a mobile app without a professional, and the testing time is reduced. However, the mentioned hearing testing technique only compares the test subject's testing data at two frequencies and does not actually test the hearing responses at other frequencies. Therefore, audiometric standards are not met, so the test results are inaccurate. When a complete hearing test is performed through a mobile app, since the test subject needs to respond to each of the single pure tones every time in the complete hearing test, the testing time would be really long. The test subject may experience fatigue or discomfort due to the long testing time, which may lead to incorrect responses, resulting in inaccurate test results.

Therefore, there is a need of providing a hearing testing system and method to obviate the drawbacks encountered from the prior art.

### SUMMARY OF THE INVENTION

It is an object of the present disclosure to provide a hearing testing system and method. In the hearing testing system and method, the user may generate a single test response corresponding to a plurality of pure tones having different decibels or frequencies. Therefore, the testing time is reduced, and the occurrence of user fatigue and erroneous responses due to excessively long testing time is reduced.

In accordance with an aspect of the present disclosure, there is provided a hearing testing system including a playback module and a detection module. The playback module includes an intensity-level adjustment module connecting to a player for setting the intensity level of a test sound, and the player plays the test sound to a test ear of a user. The test sound includes a plurality of pure tones having different decibels or frequencies. The detection module is in communication with the playback module, and the user generates a single test response corresponding to the plurality of pure tones having different decibels or frequencies. The test response is that the user inputs the number of the pure tones heard into the detection module.

In accordance with an aspect of the present disclosure, there is provided a hearing testing method, including steps of: (a) providing a playback module and a detection module communicated with each other, wherein the playback module includes an intensity-level adjustment module configured to set the intensity level of a test sound; (b) utilizing a player connected to the intensity-level adjustment module to play the test sound to a test ear of a user, wherein the test sound includes a plurality of pure tones having different decibels or frequencies; and (c) indicating the user to generate a single test response corresponding to the plurality of pure tones having different decibels or frequencies, wherein the test response is that the user inputs the number of pure tones heard into the detection module.

In accordance with another aspect of the present disclosure, there is provided a hearing testing system including a playback module and a detection module. The playback module includes an intensity-level adjustment module connecting to a player for setting the intensity level of a test sound, and the player plays the test sound to a test ear of a user. The test sound includes a plurality of pure tones having different decibels or frequencies. The detection module is in communication with the playback module, and the user generates a single test response corresponding to the plurality of pure tones having different decibels or frequencies, and inputs the test response into the detection module. The test response indicates whether any one of the plurality of pure tones in the test sound is heard by the user.

In accordance with an aspect of the present disclosure, there is provided a hearing testing method, including steps of: (a) providing a playback module and a detection module communicated with each other, wherein the playback module includes an intensity-level adjustment module configured to set the intensity level of a test sound; (b) utilizing a player connected to the intensity-level adjustment module to play the test sound to a test ear of a user, wherein the test sound includes a plurality of pure tones having different decibels or frequencies; and (c) indicating the user to generate a single test response corresponding to the plurality of pure tones having different decibels or frequencies and inputting the test response into the detection module, wherein the test response indicates whether any one of the plurality pure tones in the test sound was heard by the user.

The above contents of the present invention will become more readily apparent to those ordinarily skilled in the art after reviewing the following detailed description and accompanying drawings, in which:

### BRIEF DESCRIPTION OF THE DRAWINGS

FIG. 1 is a schematic system diagram illustrating a hearing testing system according to an embodiment of the present disclosure;

FIG. 2 is a schematic system diagram illustrating a hearing testing system according to another embodiment of the present disclosure;

FIG. 3 is a schematic audiogram output by the audiogram output module of FIG. 2.

FIG. 4 is a schematic flow chart illustrating a hearing testing method according to an embodiment of the present disclosure;

FIG. 5 is a schematic flow chart illustrating a hearing testing method according to another embodiment of the present disclosure;

FIG. 6 is a schematic flow chart illustrating step S4 of FIG. 5;

FIG. 7 is a schematic system diagram illustrating a hearing testing system according to another embodiment of the present disclosure;

FIG. 8 is a schematic flow chart illustrating a hearing testing method according to another embodiment of the present disclosure;

FIG. 9 is a schematic flow chart illustrating a hearing testing method according to another embodiment of the present disclosure; and

FIG. 10 is a schematic flow chart illustrating step S6 of FIG. 9.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

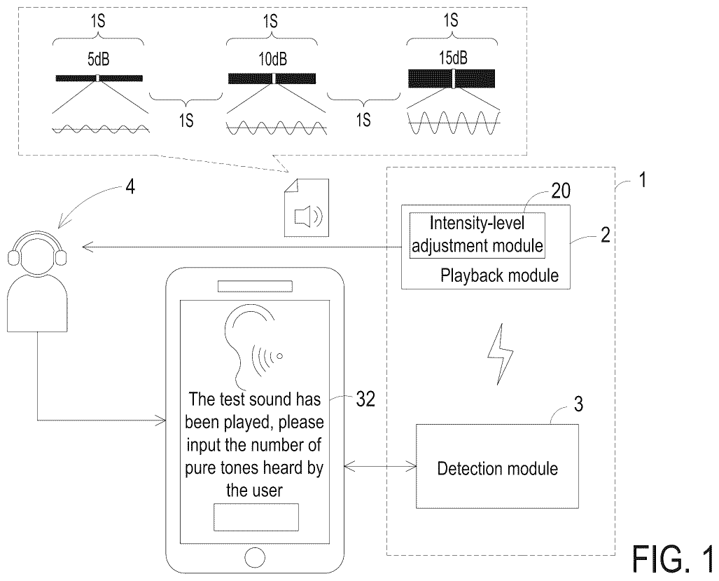

FIG. 1 is a schematic system diagram illustrating a hearing testing system 1 according to an embodiment of the present disclosure. As shown in FIG. 1, the hearing testing system 1 includes a playback module 2 and a detection module 3. In an embodiment, the playback module 2 and the detection module 3 originate from a same software program of a mobile phone or a computer. In another embodiment, the playback module 2 and the detection module 3 originate from different software programs. The playback module 2 includes an intensity-level adjustment module 20, and the intensity-level adjustment module 20 is connected to a player 4 and sets an intensity level of a test sound. The player 4 plays the test sound to a test ear of a user and is worn by the user. The player 4 is for example but not limited to a headphone or an electronic device capable of playing sound. The test sound includes a plurality of pure tones having different decibels or a plurality of pure tones having different frequencies. The detection module 3 is in communication with the playback module 2, and the user generates a single test response corresponding to the plurality of pure tones having different decibels or the plurality of pure tones having different frequencies. The test response is that the user inputs the number of the pure tones heard into the detection module 3. In the embodiment shown in FIG. 1, the test sound includes three pure tones having the same frequency but three different decibels, which are 5 dB, 10 dB and 15 dB respectively. The inter-stimulus interval and the duration of the pure tones are both 1 second. However, the decibels of each pure tone, the inter-stimulus interval, and the duration of the pure tones may be adjusted according to actual requirement and are not limited. The way for the user to generate a test response is for example but not limited to the user inputting the number of pure tones heard into a mobile app or computer software through a mobile phone touch screen or a computer keyboard. In an embodiment, the detection module 3 is a mobile app (shown in FIG. 1). After the test sound is played, the detection module 3 displays “The test sound has been played, please input the number of pure tones heard by the user” through a user interface 32. Then, the user inputs the number of pure tones heard into the detection module 3 through the user interface 32.

In the hearing testing system of the present disclosure, the user may generate a single test response corresponding to a plurality of pure tones having different decibels or a plurality of pure tones having different frequencies. Therefore, the testing time is reduced, and the occurrence of user fatigue and erroneous responses due to excessively long testing time is reduced.

The test sound of the present disclosure may include a plurality of pure tones having different decibels or a plurality of pure tones having different frequencies. In order to simplify the specification, the present disclosure only show the embodiment where the test sound includes a plurality of pure tones with different decibels. The embodiment where the test sound includes a plurality of pure tones having different frequencies is similar to the embodiment where the test sound includes a plurality of pure tones having different decibels, and the detailed description thereof is omitted herein.

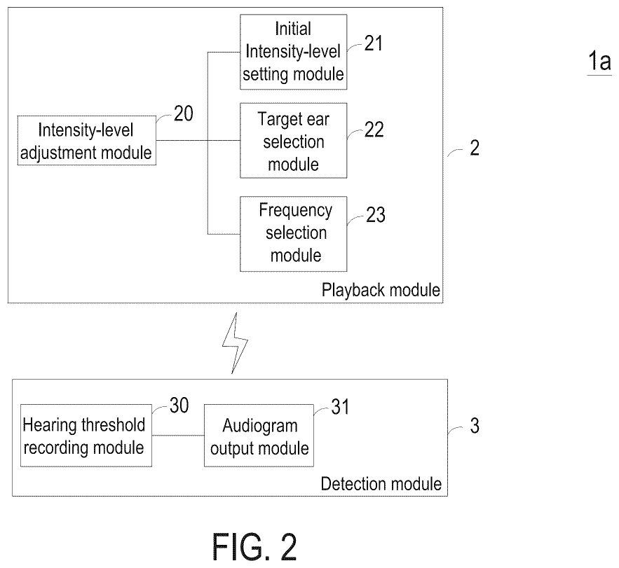

FIG. 2 is a schematic system diagram illustrating a hearing testing system 1a according to another embodiment of the present disclosure. The elements of FIG. 2 that are similar with those of FIG. 1 are represented by the same reference numerals, and the detailed description thereof is omitted herein. In the embodiment shown in FIG. 2, the playback module 2 further includes an initial intensity-level setting module 21 connected to the intensity-level adjustment module 20. The initial intensity-level setting module 21 is configured to set an initial intensity level of the test sound. The intensity-level adjustment module 20 sets the intensity levels of the plurality of pure tones of the test sound according to the initial intensity level of the test sound. The initial intensity level is for example but not limited to a fixed value, an arbitrary value, or a value generated through a setting program. For example, the setting program plays a parameter setting sound through the player 4, and the user generates a parameter setting sound response corresponding to the parameter setting sound. The parameter setting sound is for example but not limited to a piece of conversation or a piece of music at any decibel and frequency. The parameter setting sound response is that the user inputs the clarity of the conversation or music heard into the detection module 3. The way for the user to generate the parameter setting sound response is for example but not limited to the user inputting the clarity of the conversation or music heard into the mobile app or computer software through the touch screen of the mobile phone or the computer keyboard. The initial intensity-level setting module 21 sets the initial intensity level according to the parameter setting sound response.

The playback module 2 further includes a frequency selection module 23 connected to the intensity-level adjustment module 20. The frequency selection module 23 is configured to select a target frequency from a plurality of target frequencies as a test frequency. The intensity-level adjustment module 20 generates the plurality of pure tones of the test sound having different decibels corresponding to the test frequency selected by the frequency selection module 23. The user generates the single test response corresponding to the test sound with the test frequency. After the user generates the test response, the intensity-level adjustment module 20 sets the intensity levels of the plurality of pure tones of the test sound according to the test response, and the set test sound is played by the player 4. Then, the user generates the test response again corresponding to the set test sound. By utilizing the intensity-level adjustment module 20 to set the intensity levels of the pure tones of the test sounds for multiple times and to play the set test sounds through the player 4, the user may generate a plurality of test responses corresponding to the test sounds with the test frequency.

The detection module 3 further includes a hearing threshold recording module 30 and an audiogram output module 31 connected to each other. The hearing threshold recording module 30 is configured to receive the plurality of test responses corresponding to the test sounds with the test frequency and to determine a hearing threshold of the test frequency according to the plurality of test responses. The hearing threshold recording module 30 records the hearing threshold. The audiogram output module 31 is configured to generate and output an audiogram based on the recorded hearing thresholds for a plurality of different test frequencies. In an embodiment, the hearing threshold recording module 30 records the hearing thresholds of a plurality of different test frequencies, and the audiogram output module 31 is configured to generate and output an audiogram according to the hearing thresholds of the plurality of different test frequencies.

In an embodiment, when the hearing threshold recording module 30 determines the hearing threshold of the test frequency, it represents that the testing of the test frequency by the intensity-level adjustment module 20 is completed. The frequency selection module 23 selects a target frequency from the plurality of target frequencies that has not yet completed the testing as the test frequency, and the test frequency is tested through the intensity-level adjustment module 20. The hearing threshold recording module 30 records the hearing thresholds of the plurality of target frequencies, and the audiogram output module 31 generates and outputs the audiogram according to the hearing thresholds of the plurality of target frequencies. Therefore, the testing of the plurality of target frequencies is completed through the intensity-level adjustment module 20. The output format of the audiogram is for example but not limited to a line chart, a table, or a text.

In an embodiment, the playback module 2 further includes a target ear selection module 22 connected to the intensity-level adjustment module 20. The target ear selection module 22 is configured to select one ear of the user as the test ear. The target ear selection module 22 selects the ear that has not yet completed the testing of the plurality of target frequencies as the test ear among two ears of the user, and the intensity-level adjustment module 20 plays the test sound to the test ear of the user through the player 4.

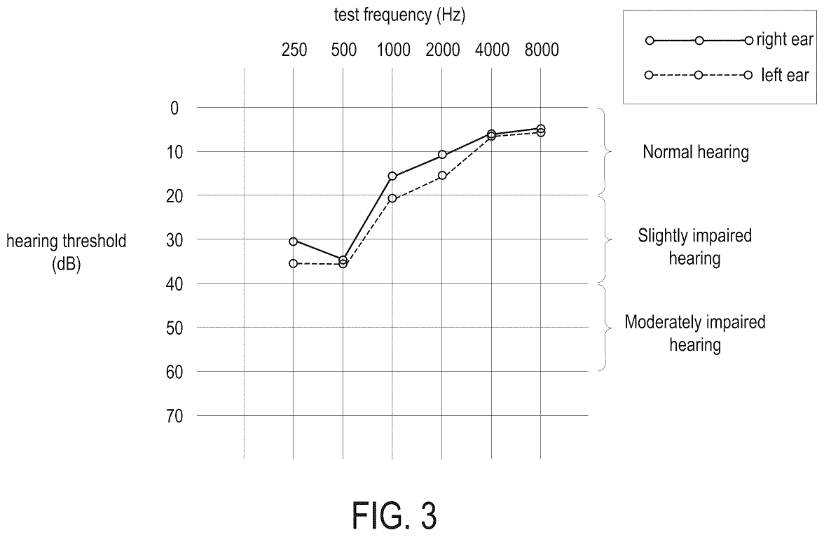

The implement of that the audiogram output module 31 generates and outputs the audiogram according to the hearing thresholds of the test frequencies is shown in FIG. 3. The audiogram shown in FIG. 3 is an example of data generated after a user undergoes a hearing testing mentioned above. The horizontal axis represents the test frequency (Hz), and the vertical axis represents the hearing threshold (dB). That is, the hearing threshold determined for the user at different test frequencies. In this embodiment, the hearing thresholds for the user's left ear at the test frequencies of 250 Hz, 300 Hz, 1000 Hz, 2000 Hz, 4000 Hz and 8000 Hz are about 35 dB, 35 dB, 20 dB, 15 dB, 5 dB and 5 dB respectively. The hearing thresholds for the user's right ear at the test frequencies of 250 Hz, 300 Hz, 1000 Hz, 2000 Hz, 4000 Hz and 8000 Hz are about 30 dB, 35 dB, 15 dB, 10 dB, 5 dB and 5 dB respectively. In an embodiment, when the hearing threshold is between 40 dB and 70 dB, it represents that the user's hearing is moderately impaired; when the hearing threshold is between 20 dB and 40 dB, it represents that the user's hearing is slightly impaired; and when the hearing threshold is between 0 dB and 20 dB, it represents that the user's hearing is normal.

The following explains how the intensity-level adjustment module 20 sets or adjusts the intensity levels of the pure tones of the test sound according to the test response and plays the set test sound through the player 4, so that the user can generate the plurality of test responses, and how the hearing threshold recording module 30 determines a hearing threshold of the test frequency according to the plurality of test responses.

The intensity-level adjustment module 20 plays the test sound to the test ear of the user through the player 4 according to the initial intensity level of the test sound set by the initial intensity-level setting module 21 and the test frequency selected by the frequency selection module 23. The test sound includes N pure tones having same test frequency but different decibels. The intensity interval between each of N pure tones is X dB, wherein N and X are positive integers. The user generates one test response corresponding to the N pure tones, and the intensity-level adjustment module 20 sets the intensity levels of the N pure tones of the test sound according to the number of pure tones heard by the user. The intensity-level adjustment module 20 controls the player 4 to play the test sound a total of M times, wherein M is greater than or equal to 2. The user generates M corresponding test responses based on M times of the test sounds, and the M test responses are stored in the detection module 3.

If the number of pure tones heard by the user is 0 when the test sound is played, the intensity-level adjustment module 20 increases the intensity level of each of the N pure tones by W1 dB, and controls the player 4 to replay the increased N pure tones to the test ear of the user, wherein W1 is a positive integer.

If the number of pure tones heard by the user is L when the test sound is played, the intensity-level adjustment module 20 decreases the intensity level of each of the N pure tones by (L−1)*W2 dB, and controls the player 4 to replay the set N pure tones to the test ear of the user, wherein L is an integer ranging from 1 to N, and W2 is a positive integer.

When a lowest-intensity pure tone heard by the user during a m-th playback of the test sound is the same as a lowest-intensity pure tone heard by the user during a (m−1)-th playback, the detection module 3 determines that the intensity level of the lowest-intensity pure tone heard by the user during the m-th playback is the hearing threshold for the test frequency, wherein m is an integer ranging from 2 to M.

In the present disclosure, since the hearing threshold for the test frequency is obtained by repeatedly increasing and decreasing the intensity levels of the pure tones based on the user's test response, this adaptive method, which is consistent with the principles of threshold determination employed in procedures such as Hughson-Westlake, helps improve the accuracy of the test results.

The following Table [1] takes N equaling 2 and X equaling 5 as an embodiment for explaining how the intensity-level adjustment module 20 sets the intensity levels of the pure tones of the test sound according to the test response and plays the set test sound through the player 4, so that the user can generate the plurality of test responses, and how the hearing threshold recording module 30 determines a hearing threshold of the test frequency according to the plurality of test responses. When N is 2 and X is 5, it represents that the test sound includes two pure tones, and the intensity interval between two pure tones is 5 dB. The intensity-level adjustment module 20 sets the intensity levels of the pure tones of the test sound played for a 1st playback by the player 4 according to the initial intensity level set by the initial intensity-level setting module 21.

TABLE 1

| | number | | number | | number |
|---|---|---|---|---|---|
| test sound | of pure | test sound | of pure | test sound | of pure |
| played for | tones | played for | tones | played for | tones |
| the 1st | heard by | the 2nd | heard by | the 3rd | heard by |
| playback | the user | playback | the user | playback | the user |
| 25 dB, | 1 | 25 dB, | 1 | hearing | |
| 30 dB | | 30 dB | | threshold = | |
| | | | | 30 dB | |
| | 2 | 20 dB, | 1 | hearing | |
| | | 25 dB | | threshold = | |
| | | | | 25 dB | |
| | 2 | 20 dB, | 2 | 15 dB, | 1 |
| | | 25 dB | | 20 dB | |
| | 0 | 35 dB, | 0 | 45 dB, | 1 |
| | | 40 dB | | 50 dB | |
| | 0 | 35 dB, | 2 | 30 dB, | 1 |
| | | 40 dB | | 35 dB | |

The pure tones of the test sound played for the 1st playback are 25 dB and 30 dB, the number of pure tones heard by the user may be 0, 1 or 2. When the number of pure tones heard by the user is 0, the intensity-level adjustment module 20 increases the intensity level of each of the two pure tones by 10(W1) dB, and the increased two pure tones are played by the player 4 again. Therefore, the pure tones of the test sound are increased from 25 dB and 30 dB to 35 dB and 40 dB respectively. Then, the player 4 plays the pure tones of the test sound for the 2nd playback at 35 dB and 40 dB. When the number of pure tones heard by the user is 1, the intensity-level adjustment module 20 decreases the intensity level of each of the two pure tones by 0*5(W2) dB, and since the decrease is 0 dB, the intensity levels of the pure tones of the test sound remain unchanged, the player 4 plays the pure tones of the test sound for the 2nd playback at 25 dB and 30 dB. When the number of pure tones heard by the user is 2, the intensity-level adjustment module 20 decreases the intensity level of each of the two pure tones by (2−1)*5(W2) dB, and the decreased two pure tones are played by the player 4 again. Therefore, the pure tones of the test sound are decreased from 25 dB and 30 dB to 20 dB and 25 dB respectively.

Please refer to the situation in Table [1] of that when the player 4 plays the test sound for the 1st and 2nd playbacks, the numbers of pure tones heard by the user are both 1. Since the lowest-intensity pure tone heard by the user when the test sound is played for the 1st playback and the lowest-intensity pure tone heard by the user when the test sound is played for the 2nd playback are both 30 dB, the detection module 3 determines that the hearing threshold of the test frequency is 30 dB.

Please refer to the situation in Table [1] that when the player 4 plays the test sound for the 1st and 2nd playbacks, the numbers of pure tones heard by the user are 2 and 1 respectively. Since the lowest-intensity pure tone heard by the user during the 1st playback of the test sound and the lowest-intensity pure tone heard by the user during the 2nd playback of the test sound are both 25 dB, the detection module 3 determines that the hearing threshold of the test frequency is 25 dB.

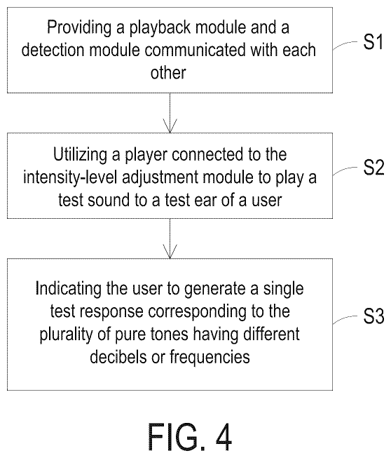

FIG. 4 is a schematic flow chart illustrating a hearing testing method according to an embodiment of the present disclosure. The hearing testing method of the present disclosure is applicable for the hearing testing systems 1 and 1a stated above. Please refer to FIGS. 1, 2 and 4. The hearing testing method of the present disclosure includes steps S1, S2 and S3. In the step S1, a playback module 2 and a detection module 3 communicated with each other are provided, and the playback module 2 includes an intensity-level adjustment module 20. In the step S2, a player 4 connected to the intensity-level adjustment module 20 is utilized to play a test sound to a test ear of a user, wherein the test sound includes a plurality of pure tones having different decibels or a plurality of pure tones having different frequencies. In the step S3, the user is indicated to generate a single test response corresponding to the plurality of pure tones having different decibels or the plurality of pure tones having different frequencies, wherein the test response is that the user inputs the number of pure tones heard into the detection module 3.

In an embodiment, the hearing testing method of the present disclosure further includes step of: utilizing an initial intensity-level setting module 21 to set an initial intensity level. The intensity-level adjustment module 20 sets the intensity levels of the plurality of pure tones of the test sound according to the initial intensity level.

In an embodiment, the hearing testing method of the present disclosure further includes steps of: utilizing a target ear selection module 22 to select one ear of the user as the test ear, wherein the target ear selection module 22 selects the ear that has not yet completed the testing of the plurality of target frequencies as the test ear among two ears of the user; and generating and outputting an audiogram by the audiogram output module 31 according to the hearing threshold of the plurality of different test frequencies when the testing of the two ears of the user is completed.

In an embodiment, the hearing testing method of the present disclosure further includes step of: utilizing the frequency selection module 23 to select a frequency from the plurality of frequencies as a test frequency.

In an embodiment, the hearing testing method of the present disclosure further includes steps of: utilizing the hearing threshold recording module 30 to receive a plurality of the test responses corresponding to the test sound with the test frequency and to determine a hearing threshold of the test frequency according to the plurality of test responses; and utilizing the hearing threshold recording module 30 to record the hearing threshold.

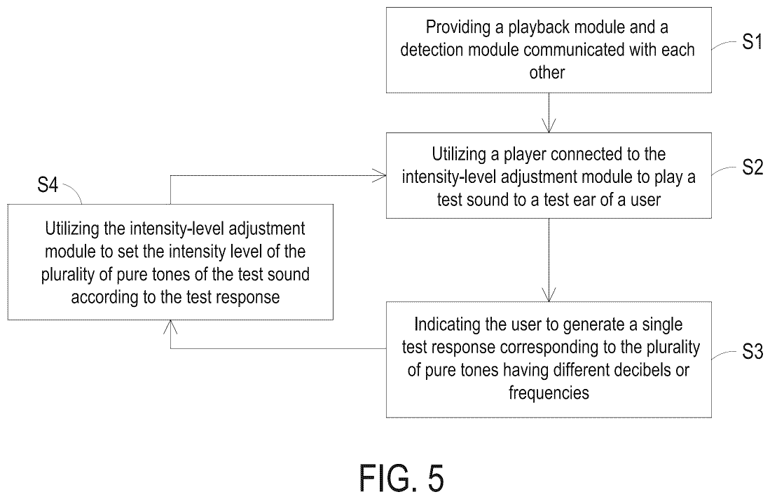

Please refer to FIGS. 2 and 5. In an embodiment, the hearing testing method of the present disclosure further includes a step S4. The step S4 is performed after the step S3. In the step S4, the intensity-level adjustment module 20 is utilized to set the intensity levels of the plurality of pure tones of the test sound according to the test response, and the step S2 is performed again after completing the step S4.

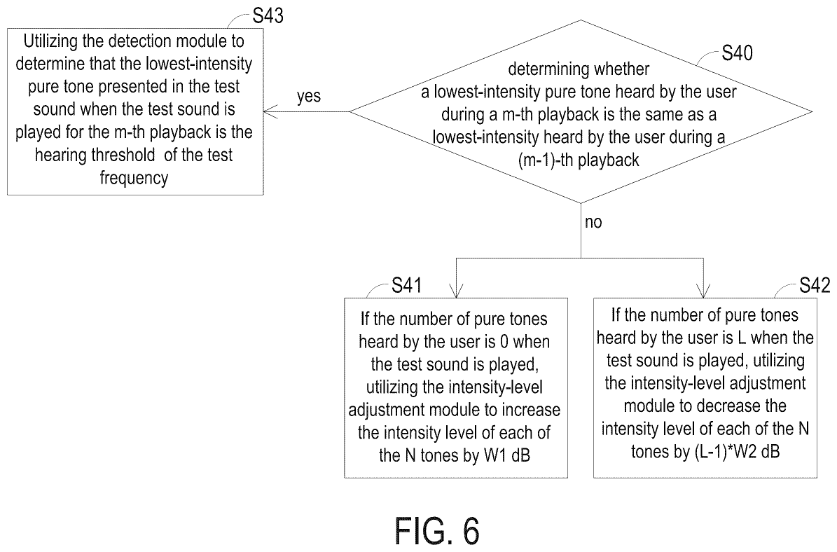

Please refer to FIGS. 2 and 6. In this embodiment, the step S4 further includes sub steps S40, S41, S42 and S43. In the step S40, the detection module 3 is utilized to determine whether a lowest-intensity pure tone heard by the user during a m-th playback is the same as a lowest-intensity pure tone heard during a (m−1)-th playback. When the determination result is satisfied (i.e., when the lowest-intensity pure tones from the m-th playback and the (m−1)-th playback are the same), the sub step S43 is performed. When the determination result is not satisfied (i.e., when the lowest-intensity pure tones from the m-th playback and the (m−1)-th playback are different), the sub step S41 or the sub step S42 is performed.

In the step S41, if the number of pure tones heard by the user is 0 when the test sound is played, the intensity-level adjustment module 20 is utilized to increase the intensity level of each of the N pure tones by W1 dB. In the step S42, if the number of pure tones heard by the user is L when the test sound is played, the intensity-level adjustment module 20 is utilized to decrease the intensity level of each of the N pure tones by (L−1)*W2 dB.

In the step S43, the detection module 3 is utilized to determine that the lowest-intensity pure tone heard by the user when the test sound is played for the m-th playback is the hearing threshold.

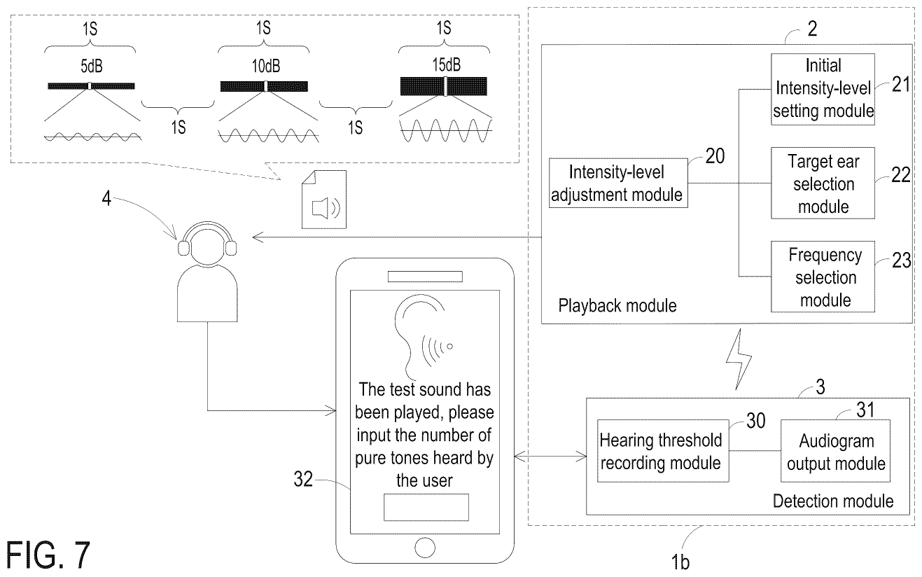

The test response is not limited to that the user inputs the number of pure tones heard into the detection module 3 as mentioned above. In an embodiment, the test response is that the user inputs a result of whether any pure tone in the test sound was heard into the detection module 3. Please refer to FIG. 7. FIG. 7 is a schematic system diagram illustrating a hearing testing system 1b according to another embodiment of the present disclosure, the structures and operations of the hearing testing system 1b are as described above, and the detailed descriptions thereof are omitted herein. In this embodiment, the player 4 plays the test sound to a test ear of a user, the test sound includes a plurality of pure tones having different decibels or a plurality of pure tones having different frequencies. The user generates a single test response corresponding to the plurality of pure tones having different decibels or the plurality of pure tones having different frequencies. The test response is that the user inputs a result of whether any pure tone in the test sound was heard into the detection module 3. In an embodiment, after the test sound is played, the detection module 3 displays “The test sound has been played, please input whether any pure tone was heard by the user” through a user interface 32. Then, the user inputs “yes” or “no” into the detection module 3 through the user interface 32, wherein “yes” represents that the user has heard at least one pure tone of the test sound, and “no” represents that the user did not hear any pure tone of the test sound.

Please refer to FIG. 7 again. The intensity-level adjustment module 20 plays the test sound to the test ear of the user through the player 4 according to the initial intensity level of the test sound set by the initial intensity-level setting module 21 and the test frequency selected by the frequency selection module 23. The test sound includes N pure tones with same test frequency but different decibels. The intensity interval between each of N pure tones is X dB, wherein N and X are positive integers. The user generates one test response corresponding to the N pure tones, and the intensity-level adjustment module 20 sets the intensity levels of the N pure tones of the test sound according to a result of whether any pure tone in the test sound was heard by the user. The intensity-level adjustment module 20 controls the player 4 to play the test sound a total of P times, wherein P is greater than or equal to 3. The user generates P corresponding test responses based on P times of the test sounds, and the P test responses are stored in the detection module 3.

If the user hears at least one pure tone when the test sound is played, the intensity-level adjustment module 20 decreases the intensity level of each of the N pure tones by W3 dB, and controls the player 4 to replay the decreased N pure tones to the test ear of the user, wherein W3 is a positive integer.

If the user did not hear any pure tone when the test sound is played, the intensity-level adjustment module 20 increases the intensity level of each of the N pure tones by N*W4 dB, and controls the player 4 to replay the set N pure tones to the test ear of the user, wherein W4 is a positive integer.

When the results of that whether any pure tone heard by the user during the (p−2)-th, (p−1)-th and p-th playbacks of the test sound are “yes”, “no” and “yes” respectively, the detection module 3 determines that a lowest-intensity pure tone presented in the test sound during the p-th playback is the hearing threshold for the test frequency, wherein p is greater than or equal to 3 and less than or equal to P.

When the results of that whether any pure tone heard by the user during the (p−2)-th, (p−1)-th and p-th playbacks of the test sound are “yes”, “yes” and “no” respectively, the detection module 3 determines that a maximum intensity pure tone presented in the test sound during the (p−1)-th playback of the test sound is the hearing threshold for the test frequency, wherein p is greater than or equal to 3 and less than or equal to P.

The following Tables [2] and [3] take N equaling 3 and X equaling 5 as an embodiment for explaining how the intensity-level adjustment module 20 sets the intensity levels of the pure tones of the test sound according to the test response and plays the set test sound through the player 4, so that the user can generate the plurality of test responses, and how the hearing threshold recording module 30 determines a hearing threshold of the test frequency according to the plurality of test responses. The test response is that the user inputs a result of whether any pure tone in the test sound was heard into the detection module 3. When N is 3 and X is 5, it represents that the test sound includes three pure tones, and the intensity interval between three pure tones is 5 dB. The intensity-level adjustment module 20 sets the intensity levels of the pure tones of the test sound played for a 1st playback by the player 4 according to the initial intensity level set by the initial intensity-level setting module 21.

TABLE 2

| test sound | | test sound | | test sound | |
|---|---|---|---|---|---|
| played for | | played for | | played for | |
| the 1st | | the 2nd | | the 3rd | |
| playback | result | playback | result | playback | result |
| 20 dB, | yes | 15 dB, | no | 30 dB, | yes |
| 25 dB, | | 20 dB, | | 35 dB, | |
| 30 dB | | 25 dB | | 50 dB | |
| | yes | 15 dB, | yes | 10 dB, | no |
| | | 20 dB, | | 15 dB, | |
| | | 25 dB | | 20 dB | |
| | no | 35 dB, | yes | 30 dB, | yes |
| | | 40 dB, | | 35 dB, | |
| | | 45 dB | | 40 dB | |
| | no | 35 dB, | yes | 30 dB, | no |
| | | 40 dB, | | 35 dB, | |
| | | 45 dB | | 40 dB | |

TABLE 3

| | | | |
|---|---|---|---|
| test sound played | | test sound played | |
| for the 4th | | for the 5th | |
| playback | result | playback | result |

| | | |
|---|---|---|
| hearing | | |
| threshold = | | |
| 30 dB | | |
| hearing | | |
| threshold = | | |
| 25 dB | | |
| 25 dB, | yes | hearing |
| 30 dB, | | threshold = |
| 35 dB | | 40 dB |
| 45 dB, | yes | hearing |
| 50 dB, | | threshold = |
| 65 dB | | 45 dB |

The pure tones of the test sound played for the 1st playback are 20 dB, 25 dB and 30 dB, the user inputs a result of whether any pure tone in the test sound was heard into the detection module 3. When the user hears at least one pure tone when the test sound is played, the intensity-level adjustment module 20 decreases the intensity level of each of the three pure tones by 5(W3) dB, and the decreased three pure tones are played by the player 4 again to the test ear of the user. Therefore, the pure tones of the test sound are decreased from 20 dB, 25 dB and 30 dB to 15 dB, 20 dB and 25 dB respectively. Then, the player 4 plays the pure tones of the test sound for the 2nd playback at 15 dB, 20 dB and 25 dB. When the user did not hear any pure tone when the test sound is played, the intensity-level adjustment module 20 increases the intensity level of each of the three pure tones by 3*5(W4) dB, and the increased three pure tones are played by the player 4 again to the test ear of the user. Therefore, the pure tones of the test sound are increased from 20 dB, 25 dB and 30 dB to 35 dB, 40 dB and 45 dB respectively. Then, the player 4 plays the pure tones of the test sound for the 2nd playback at 35 dB, 40 dB and 45 dB.

Please refer to the situation in Tables [2] and [3] that when the player 4 plays the test sound for the 1st, 2nd and 3rd playbacks, the user's responses are “yes”, “no” and “yes” respectively. Since the lowest-intensity pure tone presented in the test sound during the 3rd playback is 30 dB, the detection module 3 determines that the hearing threshold for the test frequency is 30 dB. Please refer to the situation in Tables [2] and [3] that when the player 4 plays the test sound for the 1st, 2nd and 3rd playbacks, the user's responses are “yes”, “yes” and “no” respectively. Since the maximum intensity pure tone presented in the test sound during the 2nd playback is 25 dB, the detection module 3 determines that the hearing threshold for the test frequency is 25 dB.

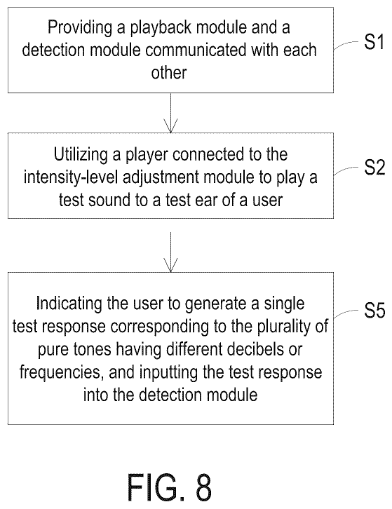

FIG. 8 is a schematic flow chart illustrating a hearing testing method according to an embodiment of the present disclosure. The hearing testing method of the present disclosure is applicable for the hearing testing systems 1, 1a and 1b stated above. Please refer to FIGS. 7 and 8. The hearing testing method of the present disclosure includes steps S1, S2 and S5. In the step S5, the user is utilized to generate a single test response corresponding to the plurality of pure tones having different decibels or the plurality of pure tones having different frequencies and to input the test response into the detection module 3, wherein the test response indicates whether any one of the plurality of pure tones in the test sound was heard by the user.

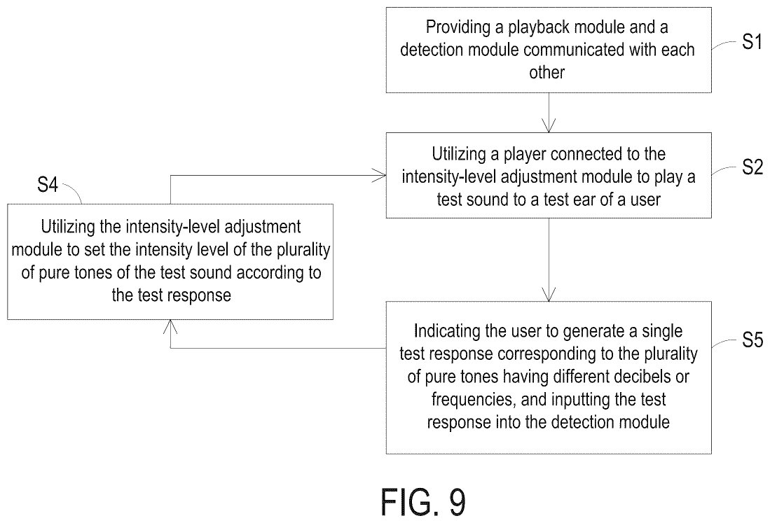

Please refer to FIGS. 7 and 9. In an embodiment, the hearing testing method of the present disclosure further includes a step S6. The step S6 is performed after the step S5. In the step S6, the intensity-level adjustment module 20 sets the intensity levels of the plurality of pure tones of the test sound according to the test response, and the step S2 is performed again after performing the step S6.

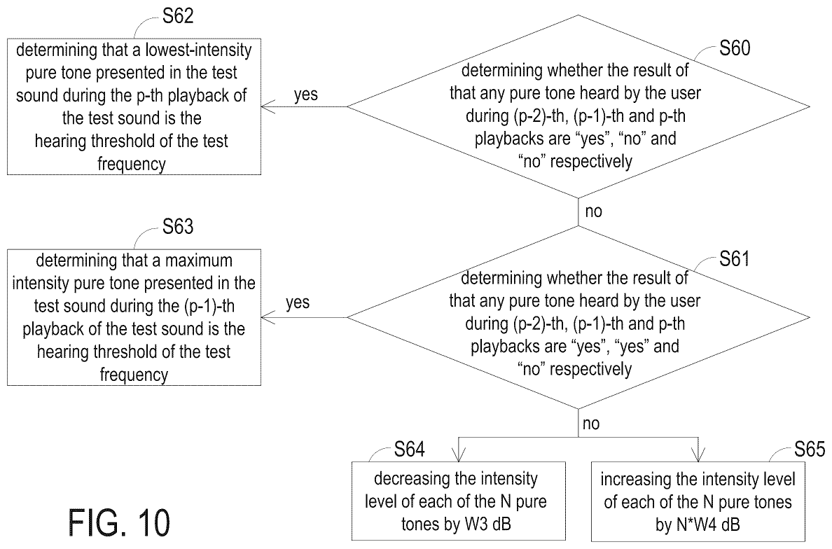

Please refer to FIGS. 7 and 10. In this embodiment, the step S6 further includes sub steps S60, S61, S62, S63 and S64. In the step S60, the detection module 3 is utilized to determine whether the result of that any pure tone heard by the user during the (p−2)-th, (p−1)-th and p-th playbacks of the test sound are “yes”, “no” and “yes” respectively. When the determination result of the step S60 is “yes”, the sub step S62 is performed. When the determination result of the step S60 is “no”, the sub step S61 is performed. In the sub step S61, the detection module 3 is utilized to determine whether the result of that any pure tone heard by the user during the (p−2)-th, (p−1)-th and p-th playbacks of the test sound are “yes”, “yes” and “no” respectively. When the determination result of the sub step S61 is “yes”, the sub step S63 is performed. When the determination result of the sub step S61 is “no”, the sub step S64 or sub step S65 is performed.

In the sub step S64, if the user hears at least one pure tone when the test sound is played, the intensity-level adjustment module 20 is utilized to decrease the intensity level of each of the N pure tones by W3 dB. In the step S65, if the user did not hear any pure tone when the test sound is played, the intensity-level adjustment module 20 is utilized to increase the intensity level of each of the N pure tones by N*W4 dB. In the sub step S62, the detection module 3 is utilized to determine that a lowest-intensity pure tone presented in the test sound during the p-th playback of the test sound is the hearing threshold of the test frequency. In the sub step S63, the detection module 3 is utilized to determine that a maximum intensity pure tone presented in the test sound during the (p−1)-th playback of the test sound is the hearing threshold of the test frequency.

From the above descriptions, the present disclosure provides a hearing testing system and method. In the hearing testing system and method of the present disclosure, the user may generate a single test response corresponding to a plurality of pure tones having different decibels or a plurality of pure tones having different frequencies. Therefore, the testing time is reduced, and the occurrence of user fatigue and erroneous responses due to excessively long testing time is reduced.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A hearing testing system, comprising:
a playback module, comprising an intensity-level adjustment module, wherein the intensity-level adjustment module is connected to a player and is configured to set an intensity level of a test sound, the player is configured to play the test sound to a test ear of a user, the test sound comprises a plurality of pure tones having different decibels or frequencies; and
a detection module, in communication with the playback module, wherein the user generates a single test response corresponding to the plurality of pure tones having different decibels or frequencies,
wherein the test response is that the user inputs the number of the pure tones heard into the detection module.

2. The hearing testing system according to claim 1, wherein the test sound comprises N pure tones having same test frequency but different decibels, and an intensity interval between each of the N pure tones is X dB, wherein N and X are positive integers.

3. The hearing testing system according to claim 2, wherein the playback module further comprises a frequency selection module connected to the intensity-level adjustment module, wherein the frequency selection module is configured to select a target frequency from a plurality of target frequencies as the test frequency, wherein the intensity-level adjustment module generates the N pure tones having different decibels corresponding to the test frequency selected by the frequency selection module.

4. The hearing testing system according to claim 3, wherein the detection module further comprises a hearing threshold recording module configured to receive a plurality of the test responses corresponding to the test sounds with the test frequency and to determine a hearing threshold of the test frequency according to the plurality of test responses, and wherein the hearing threshold recording module records the hearing threshold for the test frequency.

5. The hearing testing system according to claim 4, wherein the detection module further comprises an audiogram output module, wherein the audiogram output module is configured to generate and output an audiogram based on the recorded hearing thresholds for a plurality of test frequencies.

6. The hearing testing system according to claim 4, wherein the intensity-level adjustment module controls the player to play the test sound a total of M times, wherein M is greater than or equal to 2, and wherein the user generates M corresponding test responses based on M times of the test sounds, and the M test responses are stored in the detection module.

7. The hearing testing system according to claim 6, wherein if the number of the pure tones heard by the user is 0 when the test sound is played, the intensity-level adjustment module increases the intensity level of each of the N pure tones by W1 dB, and controls the player to replay the increased N pure tones to the test ear of the user, wherein W1 is a positive integer.

8. The hearing testing system according to claim 7, wherein if the number of the pure tones heard by the user is L when the test sound is played, the intensity-level adjustment module decreases the intensity level of each of the N pure tones by (L−1)*W2 dB, and controls the player to replay the set N pure tones to the test ear of the user, wherein L is an integer ranging from 1 to N, and W2 is a positive integer.

9. The hearing testing system according to claim 8, wherein when a lowest-intensity pure tone heard by the user during a m-th playback of the test sound is the same as a lowest-intensity pure tone heard by the user during a (m−1)-th playback, the detection module determines that the intensity level of the lowest-intensity pure tone heard by the user during the m-th playback is the hearing threshold for the test frequency, wherein m is an integer ranging from 2 to M.

10. The hearing testing system according to claim 1, wherein the playback module further comprises an initial intensity-level setting module connected to the intensity-level adjustment module, wherein the initial intensity-level setting module is configured to set an initial intensity level for the test sound, the intensity-level adjustment module sets the intensity levels of the plurality of pure tones of the test sound according to the initial intensity level.

11. The hearing testing system according to claim 1, wherein the playback module further comprises a target ear selection module configured to select one ear of the user as the test ear.

12. A hearing testing method, comprising steps of:
(a) providing a playback module and a detection module communicated with each other, wherein the playback module comprises an intensity-level adjustment module configured to set an intensity level of a test sound;
(b) utilizing a player connected to the intensity-level adjustment module to play the test sound to a test ear of a user, wherein the test sound comprises a plurality of pure tones having different decibels or frequencies; and
(c) indicating the user to generate a single test response corresponding to the plurality of pure tones having different decibels or frequencies, wherein the test response is that the user inputs the number of the pure tones heard into the detection module.

13. The hearing testing method according to claim 12, further comprising a step of utilizing an initial intensity-level setting module to set an initial intensity level, wherein the intensity-level adjustment module sets the intensity levels of the plurality of pure tones of the test sound according to the initial intensity level.

14. The hearing testing method according to claim 12, further comprising a step of utilizing a target ear selection module to select one ear of the user as the test ear.

15. The hearing testing method according to claim 12, further comprising a step of utilizing a frequency selection module to select a frequency from a plurality of frequencies as a test frequency.

16. The hearing testing method according to claim 15, further comprising steps of: utilizing a hearing threshold recording module to receive a plurality of the test responses corresponding to the test sounds with the test frequency and to determine a hearing threshold of the test frequency according to the plurality of test responses; and utilizing the hearing threshold recording module to record the hearing threshold for the test frequency.

17. The hearing testing method according to claim 16, further comprising a step (d) performed after the step (c):
(d) utilizing the intensity-level adjustment module to set intensity levels of the plurality of pure tones of the test sound according to the test response, and repeating the step (b) after completion of the step (d).

18. The hearing testing method according to claim 17, wherein the test sound comprises N pure tones with same test frequency and different decibels, and an intensity interval between each of the N pure tones is X dB, wherein N and X are positive integers, wherein the step (d) further comprises sub steps of:
(d1) utilizing the detection module to determine whether a lowest-intensity pure tone heard by the user during a m-th playback is the same as a lowest-intensity pure tone heard during a (m−1)-th playback, wherein the sub step (d4) is performed when the lowest-intensity pure tones from the m-th playback and the (m−1)-th playback are the same, and steps (d2) or (d3) are executed when the lowest-intensity pure tones from the m-th playback and the (m−1)-th playback are different;
(d2) utilizing the intensity-level adjustment module to increase the intensity level of each of the N pure tones by W1 dB if the number of pure tones heard by the user is 0 when the test sound is played;
(d3) utilizing the intensity-level adjustment module to decrease the intensity level of each of the N pure tones by (L−1)*W2 dB if the number of pure tones heard by the user is L when the test sound is played; and
(d4) utilizing the detection module to determine that the lowest-intensity pure tone presented in the test sound when the test sound is played for the m-th playback is the hearing threshold of the test frequency.

19. A hearing testing system, comprising:
a playback module, comprising an intensity-level adjustment module, wherein the intensity-level adjustment module is connected to a player and is configured to set an intensity level of a test sound, the player is configured to play the test sound to a test ear of a user, the test sound comprises a plurality of pure tones having different decibels or frequencies; and
a detection module, in communication with the playback module, wherein the user generates a single test response corresponding to the plurality of pure tones having different decibels or frequencies, and inputs the test response into the detection module, wherein the test response indicates whether any one of the plurality of pure tones in the test sound was heard by the user.
